(19) United States
(12) Patent Application Publication
(10) Pub. No.: US 2007/0264210 A1
(43) Pub. Date: Nov. 15, 2007

(54) Method of enhancing penetration of water-soluble actives

(75) Inventor: Larry Richard Robinson, Loveland, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/803,532

(22) Filed: May 15, 2007

Related U.S. Application Data
(60) Provisional application No. 60/800,378, filed on May 15, 2006.

(51) Int. Cl. A61K 31/355; A61K 8/18

(52) U.S. Cl. 424/59; 424/70.7; 424/70.12; 514/458

(57) ABSTRACT

A method of enhancing the delivery of water-soluble skin care actives into keratinous tissue, comprising the step of applying to the keratinous tissue a water-in-oil emulsion comprising an aqueous phase and a non-aqueous phase, wherein the aqueous phase comprises a water-soluble skin care active, and whereupon application of shear stress to the composition, the aqueous phase is visibly separated from the non-aqueous phase.

### CROSS REFERENCE TO RELATED APPLICATION

This application claims the benefit of U.S. Provisional Application No. 60/800,378, filed May 15, 2006.

### FIELD OF THE INVENTION

The present invention relates to personal care compositions, and methods of use thereof, which provide an enhanced delivery of water soluble actives into the skin.

### BACKGROUND OF THE INVENTION

A variety of products are available to the consumer to provide skin care benefits and to counteract what many consider undesirable “signs of skin aging,” such as fine lines, wrinkles and uneven skin texture. To be most effective, some products must be applied regularly and over an extended period of time. This may be especially important when the product is intended to provide a chronic, or long-term, benefit. To encourage frequent usage, it is important that the product have a desirable feel when applied, and also provides some indication that the product is having its intended effect (i.e. an immediate benefit). There exists a continuing need, therefore, to provide personal care compositions that provide an immediate benefit and thus encourage repeated use to provide a long-term benefit. In addition, a continuing need exists to provide compositions that more effectively deliver active ingredients into the keratinous tissue to provide a long-term benefit.

### SUMMARY OF THE INVENTION

The present invention meets the aforementioned needs, and describes compositions in the form of an emulsion which release an aqueous phase upon application of shear force, for example, by applying the composition the skin. The compositions provide a water-like, fresh feel upon application, and leave the consumer with a silky after-feel, which may encourage repeated and regular use of the product. In addition, Applicants believe that these compositions provide enhanced delivery and penetration of water soluble active ingredients into the skin. Without being limited by theory, it is believed that upon application, the aqueous phase coalesces into droplets when the composition is applied to keratinous tissue. A water soluble skin care active will be distributed predominantly into the aqueous phase, where the active may become more concentrated in the water droplets, and produce a concentration gradient that is conducive to enhancing penetration of the active into the skin. It is generally believed that enhanced penetration of many skin care actives into the skin will result in enhanced efficacy of the active.

According to one embodiment of the present invention, a method of enhancing the delivery of water-soluble skin care actives into keratinous tissue is provided, comprising the step of applying to the keratinous tissue a water-in-oil emulsion comprising an aqueous phase, a non-aqueous phase, and at least one water-soluble skin care active, whereupon application of shear stress to the composition, the aqueous phase is visibly separated from the non-aqueous phase.

According to another embodiment of the present invention, a method of enhancing delivery of skin care actives into keratinous tissue is provided, comprising the step of applying to keratinous tissue a composition comprising: from about 0.1% to about 15% of a non-emulsifying crosslinked siloxane elastomer; from about 0.1% to about 15% of an emulsifying crosslinked siloxane elastomer; from about 1% to about 40% of a solvent for the non-emulsifying and emulsifying crosslinked siloxane elastomers; a dermatologically-acceptable carrier; and at least one water soluble skin care active selected from the group consisting of vitamin B compounds, vitamin C compounds, peptides and peptide derivatives, sugar amines, oil control agents, antioxidant precursors, radical scavengers, sunscreens, protease inhibitors, skin lightening agents, a sunless tanning agent, and mixtures thereof.

### DETAILED DESCRIPTION OF THE INVENTION

The present invention describes a method of providing an immediate skin care benefit to a consumer in the form of a visible water release while providing a long-term benefit by increasing the delivery of water soluble skin care actives into keratinous tissue. The composition may be used in a variety of personal care products, non-limiting examples of which include moisturizers, conditioners, cleansers, sunscreens, anti-aging compounds, and combinations thereof. The composition may be in a variety of forms, including but not limited to an emulsion, lotion, solid, cream, gel, mousse, ointment, paste, serum, stick, etc.

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “personal care composition” means compositions suitable for topical application on mammalian keratinous tissue. “Skin care actives,” or “actives,” as used herein, means compounds that, when applied to the skin, provide a benefit or improvement to the skin. It is to be understood that skin care actives are useful not only for application to skin, but also to hair, nails and other mammalian keratinous tissue.

Herein, “stable” and “stability” mean a composition which is substantially unaltered in chemical state, physical homogeneity and/or color upon exposure to conditions reasonably expected to be incurred in shipping, storage and use, for example for a period of about 30 days at a temperature of from about 0° C. to about 40° C. Stability may be determined either by empirical observation or by appropriate methods of chemical and/or physical analysis that would be known to one of skill in the art.

“Keratinous tissue,” as used herein, refers to keratin-containing layers disposed as the outermost protective covering of mammals which includes, but is not limited to, skin, hair, nails, cuticles, etc.

“Dermatologically acceptable,” as used herein, means that the compositions or components described are suitable for use in contact with human keratinous tissue without undue toxicity, incompatibility, instability, allergic response, and the like.

“Water soluble,” as used herein, means that the skin care active is substantially dissolved in the aqueous phase and is not visually apparent with the unaided eye in a solid form such as a precipitate or a crystal. “Water soluble” is understood to include water dispersible actives. “Water dispersible” refers to actives which are suspended in the aqueous phase, but which may not be substantially dissolved.

Herein, “immediate,” means that the benefit occurs upon visual separation of the aqueous phase from the remainder of composition, as defined herein.

“Enhanced,” as used herein in reference to penetration of actives into the tissue, means that the concentration of a skin care active that is absorbed into the keratinous tissue by applying an aqueous-phase releasing composition as described herein, is statistically increased relative to the amount that is absorbed into the keratinous tissue when a substantially similar amount of a composition which comprises the same skin care active and which does not release an aqueous phase upon application is applied.

“Visibly separated,” as used herein, means that when an emulsion comprising at least two phases, an aqueous phase and a non-aqueous phase, is applied to keratinous tissue, the aqueous phase comprises individual droplets, for example having a diameter of from about 1 mm to about 1 cm, and is discemable upon the oil phase, without the aid of magnification by one having substantially unimpaired vision.

“Applied” or “application,” as used herein, means to spread the composition onto keratinous tissue with one or more fingers and/or an implement, using one continuous, unidirectional motion and light pressure, for example, as one would be expected to apply a cream to the facial skin.

Herein, “delivery enhancement device” means any device that increases the amount of active ingredient applied to and/or into the skin relative to the amount of active ingredient that is delivered without using the device.

Herein, “regulating skin condition” means improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic benefit and may include one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

Herein, “insult-affected keratinous tissue,” means keratinous tissue which exhibits discomfort, irritation, an unpleasant or irregular appearance and the like, for example after exposure to a physical and/or chemical irritant. Non-limiting examples of insult-affected keratinous tissue include sunburn and other types of burns; rashes, such as diaper rash, shaving rash and allergen-induced rashes; discoloration, such as bleaching, staining or hyperpigmentation; skin having nicks and cuts due to, for example, shaving; dry, chapped or rough skin due to exposure to example wind, cold and/or low humidity, etc. Non-limiting examples of insults include radiation, wind, low humidity, allergens, pollutants, chemical and natural irritants, bodily fluids, bodily waste, excessive moisture, bacteria, fungi, etc.

“Non-volatile,” as used herein, means materials that exhibit a vapor pressure of no more than about 0.2 mm Hg at 25° C. at one atmosphere and/or to materials that have a boiling point at one atmosphere of at least about 300° C. “Volatile,” as used herein, all materials that are not “non-volatile” as defined herein.

“Non-polar,” as used herein, means that the material has an average solubility parameter below about 6.5 (cal/cm³)^{0.5}, where “cal” means calories. Oils having a higher solubility parameter than 6.5 may be used if, when the oils are blended with other oils, the weighted average of the solubility parameter of the oil blend is below about 6.5. Herein, “weighted average” means that the volumes and the solubility parameters of the various oils are taken into account when calculating the average solubility parameter. “Polar,” as used herein means that the material has a higher average solubility parameter than non-polar compounds as defined herein. Solubility parameters are discussed extensively by C. D. Vaughan in “The Solubility Parameter: What is it?,” Cosmetics & Toiletries vol. 106, November, 1991, pp. 69-72, and also by C. D. Vaughan in “Using Solubility Parameters in Cosmetics Formulation”, 36 J. Soc. Cosmetic Chemists 319-333, September/October, 1988.

### I. Composition

The composition of the present invention is in the form of an emulsion and comprises a non-aqueous phase and an aqueous phase. Herein, the terms “non-aqueous” and “oil” are used interchangeably, as are the terms “aqueous” and “water.” Suitable types of emulsions include, but are not limited to, oil-in-water, water-in-oil, water-in-oil-in-water, and oil-in-water-in-oil emulsions. The oil may be derived from animals, plants, or petroleum, may be natural or synthetic, and may comprise silicone oils. In one embodiment, the dermatologically acceptable carrier comprises oil-in-water emulsions and water-in-oil emulsions. In one embodiment, the composition is a water-in-oil emulsion. Upon application of shear force, or shear stress, the aqueous phase is visibly separated from the oil phase and the aqueous phase may coalesce to form visible droplets within and/or upon the oil phase. The oil phase typically is substantially evenly distributed upon the skin. The aqueous phase may form visible droplets immediately upon application, and alternatively within about three seconds after application, and alternatively within about ten seconds after application.

Examples of shear force include applying to the skin, or other keratinous tissue, for example by smearing, rubbing, dabbing, wiping, etc. with a finger, hand, implement and/or a delivery enhancement device. The separate aqueous phase may provide immediate benefits, including but not limited to, an immediate indication that the product is hydrating the keratinous tissue and/or an enhanced pleasant (“silky”) feel upon application. After separation of the phases, the aqueous phase may for example be rubbed into the skin or may be allowed to evaporate.

In one embodiment, the bulk composition, prior to being applied to the keratinous tissue, is white or substantially colorless.

### A. Non-aqueous Phase

The composition may comprise from about 1.2% to about 70%, alternatively from about 5% to about 60%, and alternatively from about 10% to about 35% of a non-aqueous phase. The non-aqueous phase may comprise an emulsifying and/or non-emulsifying silicone elastomer, an elastomer solvent, one or more oil-soluble skin care actives, and mixtures thereof.

### 1. Elastomers

The composition of the present invention comprises a silicone elastomer, useful for reducing the tackiness of the composition and for providing a pleasant feel upon application. One non-limiting example of useful silicone elastomers are crosslinked organopolysiloxane (or siloxane) elastomers, as described in U.S. patent publication 2003/0049212A1. The elastomers may comprise emulsifying and non-emulsifying silicone elastomers. “Emulsifying,” as used herein, means crosslinked organopolysiloxane elastomers having at least one polyoxyalkylene (e.g., polyoxyethylene or polyoxypropylene) or polyglycerin moiety, whereas “non-emulsifying” means crosslinked organopolysiloxane elastomers essentially free of polyoxyalkylene or polyglycerin moeities.

The composition of the present invention may comprise from about 0.1% to about 15%, alternatively from about 0.1% to about 5%, and alternatively from about 0.1% to about 2% of a non-emulsifying crosslinked siloxane elastomer. In one embodiment, the non-emulsifying crosslinked siloxane elastomers are dimethicone/vinyl dimethicone crosspolymers, supplied by a variety of suppliers including Dow Corning™ (DC 9040 and DC 9041), General Electric™ (SFE 839), Shin Etsu™ (KSG-15, 16, 18 [dimethicone/phenyl vinyl dimethicone crosspolymer]), and Grant Industries (GRANSIL™ line of elastomers). Cross-linked siloxane elastomers useful in the present invention and processes for making them are further described in U.S. Pat. No. 4,970,252 to Sakuta, et al.; U.S. Pat. No. 5,760,116 to Kilgour, et al.; and U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK. In addition, suitable organopolysiloxane elastomer powders include vinyl dimethicone/methicone silesquioxane crosspolymers such as KSP-100, KSP-101, KSP-102, KSP-103, KSP-104, KSP-105 (Shin Etsu™); hybrid silicone powders comprising a fluoroalkyl group, such as KSP-200 (Shin Etsu™); and hybrid silicone powders comprising a phenyl group, such as KSP-300 (Shin EtSu™) and DC-9506 (Dow Corning™).

The composition of the present invention may comprise from about 0.1% to about 15%, alternatively from about 0.2% to about 5%, and alternatively from about 0.2% to about 2% of an emulsifying crosslinked organopolysiloxane elastomer, described in U.S. Pat. Nos. 5,412,004; 5,837,793; and 5,811,487. Non-limiting examples of suitable emulsifying elastomers include polyoxyalkylene-modified elastomers formed from divinyl compounds, e.g. siloxane polymers with at least two free vinyl groups bonded via Si-H linkages on a polysiloxane backbone. In one embodiment, the emulsifying crosslinked organopolysiloxane elastomers are dimethyl polysiloxanes crosslinked by Si-H sites on a molecularly-spherical MQ resin (R3SiO_{1/2} SiO_{4/2}), and alternatively is dimethicone copolyol crosspolymer and dimethicone, commercially available from Shin Etsu as KSG-21.

### 2. Elastomer Solvent

The composition of the present invention may comprise from about 1% to about 70%, alternatively from about 4% to about 50%, and alternatively from about 5% to about 40%, by weight of the non-aqueous phase, of a suitable solvent for the crosslinked organopolysiloxane elastomers. Non-limiting examples of suitable solvents are described in U.S. patent publication 2003/0049212A1. The concentration of the solvent in the cosmetic compositions of the present invention may vary depending upon the type and amount of solvent and the cross-linked siloxane elastomer employed, and when combined with the cross-linked organopolysiloxane elastomer particles of the present invention, suspends and swells the elastomer particles to provide an elastic, gel-like network or matrix. The carrier for the cross-linked siloxane elastomer is liquid under ambient conditions, and in one embodiment has a low viscosity to provide for improved spreading on the skin.

The solvent may comprise volatile, non-polar oils; non-volatile, polar oils; non-volatile, non-polar oils; and non-volatile paraffinic hydrocarbon oils. Non-limiting examples of suitable non-polar, volatile oil are disclosed in U.S. Pat. No. 4,781,917 issued to Luebbe et al. and include polydecanes such as isododecane and isodecane (e.g., Permethyl-99A, available from Presperse™ Inc.) and C7-C15 isoparaffins (e.g. the Isopar Series, from Exxon™ Chemicals); cyclomethicones of varying viscosities, e.g., Dow Corning™ 200, Dow Corning™ 244, Dow Corning™ 245, Dow Corning™ 344, and Dow Corning™ 345, Silicone Fluids, commercially available from G. E. Silicones, (e.g. SF-1204, SF-1202, GE 7207 and GE 7158); and SWS-03314 (commercially available from SWS Silicones™ Corp.).

Polar, non-volatile oils useful in the present invention include, but are not limited to, silicone oils; hydrocarbon oils; fatty alcohols; fatty acids; esters of mono and dibasic carboxylic acids with mono and polyhydric alcohols; polyoxyethylenes, polyoxypropylenes, mixtures of polyoxyethylene and polyoxypropylene ethers of fatty alcohols; and mixtures thereof. In one embodiment, the polar, non-volatile oil is selected from the group consisting of propoxylated ethers of C14-C18 fatty alcohols having a degree of propoxylation below about 50, esters of C2 -C8 alcohols and C12-C26 carboxylic acids (e.g. ethyl myristate, isopropyl palmitate), esters of C12-C26 alcohols and benzoic acid (e.g. Finsolv™ TN supplied by Finetex™), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof.

Examples of suitable non-volatile, non-polar oils include, but are not limited to non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of useful oils include Viscasil™ series (General Electric); the Dow Coming 200 series (Dow Coming Corp.); SF 1075 methyl-phenyl fluid (General Electric) and 556 Cosmetic Grade Fluid (Dow Corning Corp.).

Non-volatile paraffinic hydrocarbon oils useful in the present invention are described in U.S. Pat. No. 5,019,375 issued to Tanner et al. and in 2003/0049212A1, and include mineral oils and branched-chain hydrocarbons such as Permethyl™ 102A, 103A and 104A (Permethyl Corporation); and Ethylflo™ 364 (Ethyl Corp.). Additional suitable solvents useful herein are described in U.S. Pat. No. 5,750,096 to Guskey et al.

### B. Aqueous Phase

The composition of the present invention comprises an aqueous phase. In one embodiment the composition comprises from about 25% to about 98.8%, alternatively from about 40% to about 95%, and alternatively from about 65% to about 90% of the aqueous phase. The aqueous phase may in turn comprise an additional emulsifier, one or more water-soluble skin care actives, and mixtures thereof.

### 1. Additional Emulsifier

The composition of the present invention may contain an additional emulsifier, useful for dispersing and suspending the aqueous phase within the oil phase in a water-in-oil emulsion. The composition may comprise from about 0.001% to about 5%, alternatively from about 0.01% to about 5% alternatively from about 0.1% to about 3%, and alternatively from about 0.1% to about 2%, of at least one additional emulsifier.

A wide variety of emulsifying agents can be employed herein to form a water-in-silicone emulsion, and are described in U.S. patent publication 2003/0049212A1. In one embodiment, the additional emulsifiers are silicone emulsifiers, including organically modified organopolysiloxanes (silicone surfactants) such as dimethicone copolyols. Examples of commercially available dimethicone copolyols useful herein are Dow Corning® 190, 193, Q2-5220, 2501 Wax, 2-5324 fluid, and 3225C; ABIL™ EM-90, ABIL™ WE-09 and ABIL® WS-08 (Goldschmidt), KF-6028 and KF-6106 (Shin-Etsu™).

In one embodiment, the additional emulsifier is a non-silicone emulsifier, non-limiting examples of which include non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

### 2. Actives

The composition of the present invention comprises at least one water-soluble skin care active and may comprise at least one additional oil-soluble skin care active, both useful for regulating and/or improving the condition of mammalian skin. Solubility in water and oil is within the knowledge of one of skill in the art, and can be determined using known methods of analysis. One of skill in the art further will understand that solubility may be affected by the type and concentration of other components in the composition, and other conditions such as pH, ionic strength, etc. Many skin care actives may provide more than one benefit, or operate via more than one mode of action; therefore, classifications herein are made for the sake of convenience and are not intended to limit the active to that particular application or applications listed.

### Vitamins

The compositions of the present invention may comprise from about 0.0001% to about 50%, alternatively from about 0.001% to about 10%, alternatively from about 0.01% to about 5%, and alternatively from about 0.1% to about 1%, of one or more vitamins. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

### Peptides and Peptide Derivatives

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins (Ridulisse C™, from Silab, France), carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

### Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

### Sunscreens

The compositions of the subject invention may comprise one or more sunscreen actives (or sunscreen agents) and/or ultraviolet light absorbers. Herein, “sunscreen active” includes both sunscreen agents and physical sunblocks. Sunscreen actives and ultraviolet light absorbers may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxy-cinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy) propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquatemium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimid-azole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof. In one embodiment, the composition comprises from about 1% to about 20%, and alternatively from about 2% to about 10% by weight of the composition, of the sunscreen active and/of ultraviolet light absorber. Exact amounts will vary depending upon the chosen sunscreen active and/or ultraviolet light absorber and the desired Sun Protection Factor (SPF), and are within the knowledge and judgment of one of skill in the art.

### Oil control agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, resorcinol, sulfur, erythromycin, zinc, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

### Flavonoids

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773, issued to Bissett, and include, but are not limited to, unsubstituted flavanones, methoxy flavanones, unsubstituted chalcones, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin and glucosyl hesperidin, isoflavones such as soy isoflavones, including but not limited to genistein, daidzein, quercetin, and equol, their glucosyl derivatives, 2′,4-dihydroxy chalcone, and mixtures thereof.

The compositions of the present invention may comprise from about 0.01% to about 20%, alternatively from about 0.1% to about 10%, and alternatively from about 0.5% to about 5% of flavonoids.

### Skin Lightening Agents

The present compositions may comprise from about 0.1% to about 10%, and alternatively from about 0.2% to about 5%, of a skin lightening agent. The skin lightening agent may improve the appearance of the skin and/or reduce hyperpigmentation. Useful whitening agents useful herein include azelaic acid, butyl hydroxy anisole, gallic acid, hydroquinoine, kojic acid, arbutin and deoxy-arbutin, mulberry extract, undecylenoyl phenylalanine, octadecenedioic acid, octadecenedioic acid, salts and derivatives of any of the foregoing, and mixtures thereof.

### Other Skin Care Actives

The compositions of the present invention further may comprise non-vitamin antioxidants and radical scavengers, minerals, preservatives, hair growth regulators, phytosterols and/or plant hormones, protease inhibitors, tyrosinase inhibitors, and anti-inflammatory agents.

Suitable non-vitamin antioxidants and radical scavengers include, but are not limited to, BHT (butylated hydroxy toluene), butylated hydroxy benzoic acids, L-ergothioneine (available as THIOTANE™), tetrahydrocurcumin, cetyl pyridinium chloride, diethylhexyl syrinylidene malonate (available as OXYNEX™), 6-hydroxy-2,5,7,8-tetramethylchroman-2-carboxylic acid (available as TroloX™), hexadec-8-ene-1,16-dicarboxylic acid (octadecene dioic acid; available as ARLATONE™ Dioic DCA from Uniqema), ubiquinone (co-enzyme Q10), tea extracts including green tea extract, yeast extracts or yeast culture fluid (e.g., Pitera™), gallic acid, uric acid, sorbic acid, lipoic acid, amines (e.g., N,N-diethylhydroxylamine, amino-guanidine), sulfhydryl compounds including glutathione, dihydroxy fumaric acid, lycine pidolate, arginine pilolate, nordihydroguaiaretic acid, curcumin, lysine, methionine, proline, superoxide dismutase, silymarin, grape skin/seed extracts, melanin, rosemary extracts, salts and derivatives of any of the foregoing, and combinations thereof.

Suitable examples of hair growth regulators include, but are not limited to hexamidine, butylated hydroxytoluene (BHT), hexanediol, panthenol and pantothenic acid derivates, their isomers, salts and derivatives, and mixtures thereof.

Suitable minerals include zinc, manganese, magnesium, copper, iron, selenium and other mineral supplements. “Mineral” is understood to include minerals in various oxidation states, mineral complexes, salts, derivatives, and combinations thereof.

Suitable examples of plant sterols (phytosterols) and/or plant hormones include, but are not limited to, sitosterol, stigmasterol, campesterol, brassicasterol, kinetin, zeatin, and derivatives and mixtures thereof.

Suitable protease inhibitors include, but are not limited to, hexamidine, vanillin acetate, menthyl anthranilate, soybean trypsin inhibitor, Bowman-Birk inhibitor, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to nonsteroidal anti-inflammatory agents (NSAIDS), including but not limited to ibuprofen, naproxen, flufenamic acid, etofenamate, aspirin, mefenamic acid, meclofenamic acid, piroxicam and felbinac; glycyrrhizic acid (also known as glycyrrhizin, glycyrrhixinic acid, and glycyrrhetinic acid glycoside), glycyrrhetenic acid, other licorice extracts; candelilla wax, bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, red clover extract, and sea whip extract, derivatives of any of the foregoing, and mixtures thereof.

Other useful skin care actives include moisturizing and/or conditioning agents, such as glycerol, petrolatum, aloe vera, allantoin, bisabolol, dipotassium glycyrrhizinate, and urea; dehydroepiandrosterone (DHEA), its analogs and derivatives; exfoliating agents, including alpha- and beta-hydroxyacids, alpha-keto acids, glycolic acid and octanoyl salicylate; desquamation actives, including zwitterionic surfactants; antimicrobial agents; anti-cellulite agents, such as caffeine, theophylline, theobromine, and aminophylline; antidandruff agents such as piroctone olamine, 3,4,4′-trichlorocarbanilide (trichlosan), triclocarban and zinc pyrithione; dimethyl aminoethanol (DMAE); creatine; (sunless) tanning agents, such as dihydroxy acetone (DHA); chelators, for example, furildioxime and furilmonoxime; dialkanoyl hydroxyproline compounds; soy extracts, such as soybean milk, soybean paste, and miso salts; amino acids; olive oil derivatives such as Sodium PEG-7 Olive Oil Carboxylate, topical anaesthetics, such as benzocaine, lidocaine, bupivacaine, chlorprocaine, dibucaine, etidocaine, mepivacaine, tetracaine, dyclonine, hexylcaine, procaine, cocaine, ketamine, pramoxine, phenol; salts and derivatives of any of the foregoing; and mixtures thereof.

### C. Other ingredients

### Thickening Agents

The compositions of the present invention may comprise from about 0.1% to about 5%, alternatively from about 0.1% to about 4%, and alternatively from about 0.25% to about 3%, of a thickening agent. Nonlimiting classes of thickening agents include but not limited to carboxylic acid polymers, crosslinked polyacrylate polymers, polyacrylamide polymers, polysaccharides, gums and mixtures thereof.

### II. Methods of Use

The present invention describes a method of regulating the condition of mammalian skin, of signaling an immediate, or acute, benefit to a consumer and of increasing the penetration of water soluble skin care actives into the keratinous tissue. The method comprises the step of topically applying to mammalian skin a personal care composition described herein. Alternatively, the method may comprise the step of applying the composition described herein to insult-affected keratinous tissue, to regulate and/or improve the condition of such tissue, and/or to provide relief from the effects of the insult.

The composition may be applied to any keratinous tissue, including keratinous tissue in need of one or more benefits. Benefits include regulating and/or improving the condition of keratinous tissue, non-limiting examples of which include reducing the appearance of wrinkles, reducing the appearance of deep lines, reducing the appearance of fine lines, reducing the appearance of large pores, reducing the thickness of keratinous tissue, increasing the convolution of the dermal-epidermal border, increasing elasticity, reducing the appearance of cellulite, reducing the appearance of discoloration, reducing the appearance of hyperpigmentation, reducing the appearance of under-eye circles, reducing the appearance of sallowness, and combinations thereof. Alternatively, the benefit may include reducing wrinkles, reducing deep lines, reducing fine lines, reducing large pores, reducing cellulite, reducing hyperpigmentation, reducing undereye circles, reducing puffiness, and combinations thereof.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. In one embodiment, the composition is gently spread onto the skin to facilitate the separation of the aqueous phase from the oil-phase. When the aqueous phase has separated and coalesced into visibly enhanced droplets, the composition may be left as is on the keratinous tissue. Alternatively, the composition allowed to remain on the skin for 5 seconds, 10 seconds, 30 seconds, or 1 minute prior to being rubbed into the keratinous tissue.

The amount of the composition applied, the frequency of application and the period of use will vary widely depending upon the level of components of a given composition and the level of regulation desired. For example, from about 0.01 g composition/cm² to about 1 g composition/cm² of keratinous tissue may be applied. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

### EXAMPLES 1-6

The following are non-limiting examples of compositions that may be applied to keratinous tissue in accordance with the methods described herein.

| | | | | | | |
|---|---|---|---|---|---|---|
| | EX 1 | EX 2 | EX 3 | EX 4 | EX 5 | EX 6 |
| Ingredient | (Wt %) | (Wt %) | (Wt %) | (Wt %) | (Wt %) | (Wt %) |

Phase A

| | | | | | | |
|---|---|---|---|---|---|---|
| Dimethicone | 4.0 | 4.0 | 6.0 | 3.0 | 4.0 | 4.0 |
| Polymethylsilsesquioxane *1 | 4.0 | 4.0 | 6.0 | — | 4.0 | 4.0 |
| DC9040 *2 | 3.0 | 3.0 | 4.5 | — | — | 3.0 |
| DC9045 *3 | — | — | — | — | 3.0 | — |
| KSG-15 *4 | — | — | — | 2.5 | — | — |
| Cyclopentasiloxane | 3.0 | 3.0 | 6.0 | — | 3.0 | 3.0 |
| KSG-210 *5 | 2.5 | 5.0 | 4.0 | 5.0 | 2.75 | 2.75 |
| KF-6028 *6 | — | — | 0.15 | — | — | — |
| KF-6017 *7 | — | — | — | 0.3 | — | — |
| Cover Leaf AR-80 5% | — | — | 5.0 | — | — | — |
| KF-9901 *8 | | | | | | |
| KSG-18 *9 | — | — | — | 1.5 | — | — |
| Isopropyl Isostearate | — | — | — | 2.2 | — | — |
| Fragrance | 0.1 | 0.1 | 0.1 | — | — | — |

Phase B

| | | | | | | |
|---|---|---|---|---|---|---|
| Glycerin, USP | 10.0 | 10.0 | 30.0 | 5.0 | 7.0 | 10.0 |
| Niacinamide *10 | 1.0 | — | 5.0 | — | 3.0 | 4.0 |
| Elastab HP100 *11 | — | — | — | — | 0.1 | 0.1 |
| Pentylene Glycol | 2.0 | 2.0 | 2.0 | 3.0 | — | 3.0 |
| 1,2-Hexane Diol | — | — | — | — | 3.0 | — |
| Sodium Chloride | 0.5 | 0.5 | 0.5 | 0.5 | 0.5 | 0.5 |
| Panthenol | 0.5 | 0.5 | 0.5 | — | 1.0 | 1.0 |
| Methylparaben | 0.2 | 0.2 | 0.2 | 0.2 | — | 0.2 |
| Sodium Citrate | 0.2 | 0.2 | 0.2 | 0.2 | — | 0.2 |
| Citric Acid | 0.03 | 0.03 | 0.03 | — | — | 0.03 |
| Sodium Benzoate | 0.07 | 0.07 | 0.07 | 0.07 | — | 0.07 |
| Ethylparaben | 0.05 | 0.05 | 0.05 | 0.05 | — | 0.05 |
| Benzyl Alcohol | — | — | — | 0.2 | — | — |
| Glydant Plus *12 | — | — | — | — | 0.3 | — |
| N-Acetyl Glucosamine | — | — | 2.0 | 5.0 | — | — |
| Ascorbyl Glucoside | — | 2.0 | — | — | 1.0 | — |
| Disodium EDTA | — | — | — | 0.1 | — | 0.1 |
| Water | q.s. to | q.s. to | q.s. to | q.s. to | q.s. to | q.s. to |
| | 100 | 100 | 100 | 100 | 100 | 100 |

*1 E.g., Tospearl 145 A or CF 600. Available from GE Toshiba Silicone
*2 12.5% Dimethicone Crosspolymer in Cyclopentasiloxane. Available from Dow Corning
*3 12.5% Dimethicone in Cyclopentasiloxane. Available from Dow Corning
*4 5% Dimethicone/Vinyl Dimethicone Crosspolymer in Dimethicone. Available from Shin-Etsu
*5 25% Dimethicone PEG-10/15 Crosspolymer in Dimethicone. Available from Shin-Etsu
*6 PEG-9 Polydimethylsiloxyethyl Dimethicone. Available from Shin-Etsu
*7 PEG-10 Dimethicone. Available from Shin-Etsu
*8 Silica, Alumina, Titanium Dioxide, Talc with surface-coat by Dimethicone/Methicone Copolymer. Available in Catalysts & Chemicals
*9 25% Dimethicone/Vinyl Dimethicone Crosspolymer in Dimethicone. Available from Shin-Etsu
*10 Additionally or alternatively, the composition may comprise one or more other skin care actives, their salts and derivatives, as disclosed herein, in amounts also disclosed herein as would be deemed suitable by one of skill in the art.
*11 Hexamidine diisethionate, availabile from Laboratoires Serobiologiques.
*12 DMDM Hydantoin, Iodopropynyl butylcarbamate, 1,3 butylenel glycol in water. Available from Lonza Inc.

In a suitable container, combine the ingredients of Phase A. In a separate suitable container, combine the ingredients of Phase B. Mix each phase using a suitable mixer (e.g., Anchor blade, propeller blade, IKA T25) until each phase is homogenous. Slowly add Phase B to Phase A while continuing to mix Phase A. Continue mixing until batch is uniform. Pour product into suitable containers and store at room temperature.

### EXAMPLE 7

The following example describes how insult-affected keratinous tissue may be regulated and/or improved by application of a suitable composition. These examples are for illustrative purposes only, and are not intended to limit the type of active that may be applied to a particular insult-affected area of skin. All actives are in a water-soluble form.

Apply a composition described below, in an amount of approximately 0.1 g of composition per cm², to an area of insult-affected skin. Wipe the composition onto the skin until visibly distinct droplets appear. Alternatively, the composition may be dabbed onto the affected area with an implement, such as a swab or stick applicator to produce visibly distinct droplets. Allow the composition to remain on the skin for approximately 1 minute. The composition may then be further rubbed into the skin.

| Composition | Additional Water-Soluble Active | Insult |
|---|---|---|
| Example 2 | Anti-inflammatory agent, e.g. | Sunburn, burns |
| | glycyrrhetenic acid | |
| Example 3 | Skin lightening agent, such as | Hyperpigmentation |
| | Undecylenoyl phenylalanine | |
| Example 1 | Peptide, e.g. palmitoyl-KTTKS; | UV-damage, e.g. lines, |
| | sunscreen; antioxidants | wrinkles, dry and/or |
| | | peeling skin |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of enhancing the delivery of water-soluble skin care actives into keratinous tissue, comprising the step of applying to the keratinous tissue a composition comprising an water-in-oil emulsion comprising a aqueous phase and a non-aqueous phase, wherein the aqueous phase comprises a water-soluble skin care active, and whereupon application of shear stress to the composition, the aqueous phase is visibly separated from the non-aqueous phase, as evidenced by the visual presence of water droplets on the keratinous tissue.

2. The method of claim 1, wherein the composition comprises from about 1.2% to about 70% of the non-aqueous phase.

3. The method of claim 1, wherein the non-aqueous phase comprises an emulsifying crosslinked siloxane elastomer, a non-emulsifying crosslinked siloxane elastomer, and mixtures thereof.

4. The method of claim 3, wherein the non-aqueous phase comprises from about 0.1% to about 15% of the emulsifying crosslinked siloxane elastomer.

5. The method of claim 3, wherein the non-aqueous phase comprises from about 0.1% to about 15% of the non-emulsifying crosslinked siloxane elastomer.

6. The method of claim 3, wherein the composition comprises from about 1% to about 70%, by weight of the non-aqueous phase, of an elastomer solvent.

7. The method of claim 1, wherein the non-aqueous phase further comprises an oil-soluble skin care active.

8. The method of claim 7, wherein the oil-soluble skin care active is selected from the group consisting of vitamin E compounds, sunscreens, ultraviolet light absorbers, and combinations thereof.

9. The method of claim 1, wherein the water-soluble skin care active is selected from the group consisting of vitamin B compounds, vitamin C compounds, peptides and peptide derivatives, sugar amines, oil control agents, antioxidant precursors, radical scavengers, sunscreens, protease inhibitors, skin lightening agents, a sunless tanning agent, and mixtures thereof.

10. The method of claim 1, wherein the water-soluble skin care active is selected from the group consisting of niacinamide, an ascorbyl glucoside, N-acetyl glucosamine, dihydroxyacetone, a pentapeptide, hexamidine, sodium dehydroacetate, hydroquinone, undecylenoyl phenylalanine, cetyl pyridinium chloride, dihydroxyacetone, salts and derivatives thereof, and mixtures thereof.

11. The method of claim 1, wherein the aqueous phase comprises from about 0.001% to about 5% of at least one additional emulsifier.

12. The method of claim 11, wherein the additional emulsifier is a silicone emulsifier.

13. The method of claim 11, wherein the additional emulsifier is a non-silicone emulsifier.

14. A method of providing to a consumer an immediate benefit and of enhancing delivery of a skin care active into keratinous tissue, comprising the step of applying to keratinous tissue in need of a benefit a composition comprising from about 1.2% to about 70% of a non-aqueous phase and from about 30% to about 98.8% of a aqueous phase, wherein:
a) the non-aqueous phase comprises:
i. from about 0.1% to about 15%, by weight of the composition, of a non-emulsifying crosslinked siloxane elastomer;
ii. from about 0.1% to about 15%, by weight of the composition, of an emulsifying crosslinked siloxane elastomer;
iii. from about 1% to about 70%, by weight of the non-aqueous phase, of a solvent for the non-emulsifying and emulsifying crosslinked siloxane elastomers;
b) the aqueous phase comprises and at least one water-soluble skin care active selected from the group consisting of vitamin B compounds, vitamin C compounds, peptides and peptide derivatives, sugar amines, oil control agents, antioxidant precursors, radical scavengers, sunscreens, protease inhibitors, skin lightening agents, a sunless tanning agent, and mixtures thereof, and a dermatologically-acceptable carrier.

15. The method of claim 14, wherein the composition further comprises an oil-soluble skin care active selected from the group consisting of vitamin E compounds, sunscreens, ultraviolet light absorbers, and combinations thereof.

16. The method of claim 14, wherein the immediate benefit is the appearance of droplets of water.

17. The method of claim 14, wherein the skin care active provides a chronic benefit.

18. The method of claim 17, wherein the chronic benefit is selected from the group consisting of reducing signs of aging, reducing the appearance of wrinkles, reducing the appearance of deep lines, reducing the appearance of fine lines, reducing the appearance of large pores, reducing the thickness of keratinous tissue, increasing the convolution of the dermal-epidermal border, increasing elasticity, reducing the appearance of cellulite, reducing the appearance of discoloration, reducing the appearance of hyperpigmentation, reducing the appearance of under-eye circles, reducing the appearance of sallowness, and combinations thereof.

19. The method of claim 14, wherein the keratinous tissue is insult-affected mammalian skin.

20. The method of claim 19, wherein the insult-affected mammalian skin is selected from the group consisting of burned, sunburned, rash-affected, diaper rash-affected, shaving rash-affected, allergen-induced rash-affected, bleached, stained, hyperpigmented; skin having nicks, skin having cuts, dry skin, rough skin, and combinations thereof.
